(19) United States
(12) Patent Application Publication
(10) Pub. No.: US 2022/0184096 A1
(43) Pub. Date: Jun. 16, 2022

(54) APPLICATIONS OF IMINOSTILBENE IN TERMS OF PREVENTING AND TREATING CARDIAC CEREBRAL ISCHEMIA/REPERFUSION INJURY

(71) Applicant: CHINESE ACADEMY OF MEDICAL SCIENCES INSTITUTE OF MEDICINAL PLANT DEVELOPMENT, Beijing (CN)

(72) Inventors: Xiaobo SUN, Beijing (CN); Guibo SUN, Beijing (CN); Shan LU, Beijing (CN); Xudong XU, Beijing (CN); Yu TIAN, Beijing (CN)

(21) Appl. No.: 17/438,262

(22) PCT Filed: Dec. 29, 2020
(86) PCT No.: PCT/CN2020/140577

§ 371 Date: Sep. 10, 2021

(30) Foreign Application Priority Data
Dec. 30, 2019 (CN) 201911393436.0

(51) Int. Cl. A61K 31/55; A61P 9/10

(52) U.S. Cl. CPC A61K 31/55; A61P 9/10

(57) ABSTRACT

Applications of iminostilbene in preparing a medicament for treating a cardiovascular disease, specifically, cardiac cerebral ischemia/reperfusion injury. Iminostilbene is capable of reducing three myocardial enzymes and inflammatory factors during ischemia reperfusion and reduces apoptosis during ischemia reperfusion.

### TECHNICAL FIELD

The present invention relates to the field of cardiac-cerebral vascular disease, and more particularly, the present invention relates to applications of iminostilbene in preventing and treating cardiac cerebral ischemia-reperfusion injury.

### BACKGROUND OF THE INVENTION

With the accelerating process of social aging and urbanization, and popular of unhealthy lifestyle, people are generally exposed to risk factors of cardiac-cerebral vascular disease, and thus cardiac-cerebral vascular disease has become one of the most frequently occurring diseases. Ischemic cardiovascular and cerebral vascular diseases caused by reasons such as thrombus, blood vessel rupture, etc. are very common, and generally treated with anti-thrombotic drugs to remove extravasated blood. After the recovery of blood reperfusion, the ischemic myocardium and brain will suffer from further aggravation in various aspects, that is, ischemia-reperfusion injury, which becomes a major obstacle in the treatment of ischemic diseases. The causes of reperfusion injury are not yet fully clear, and free radical accumulation, cell calcium overload, membrane damage, etc. may all be the causes of the ischemia reperfusion injury. Currently, the treatment methods and drugs are still limited, and the effect needs to be improved.

Iminostilbene (ISB) is a dibenzazepine compound, which is currently only used for anti-hepatitis or used as an intermediate in the synthesis of anti-epileptic drug carbamazepine. In addition, iminostilbene is only used in tail gas catalysts and transistors. There is no report on its use in the prevention and treatment of cardiac cerebral ischemia-reperfusion injury.

### SUMMARY OF THE INVENTION

In order to develop new types of drugs for treating cardiac cerebral ischemia-reperfusion injury, we carry out study and find that iminostilbene has protective effects against myocardium and cerebral ischemia, and further explore the action mechanism of the above protective effects of iminostilbene, which provides experimental data and theoretical basis for the development of iminostilbene in treatment of cardiovascular and cerebrovascular diseases.

In one aspect, the present application provides use of iminostilbene in the manufacture of a medicament for treating a cardiac-cerebral vascular disease.

Further, the cardiac-cerebral vascular disease is ischemia-reperfusion injury.

Further, the cardiac-cerebral vascular disease is cerebral ischemia-reperfusion injury.

Further, the iminostilbene reduces cerebral infarction area.

Further, the cardiac-cerebral vascular disease is myocardial ischemia-reperfusion injury.

Further, the cardiac-cerebral vascular disease is ischemic heart disease.

Further, the iminostilbene reduces myocardial infarction area, and/or reduces LDH, AST and CK levels, and/or reduces inflammatory factors, and/or reduces myocardial apoptosis.

In another aspect, the present application provides use of iminostilbene in the manufacture of a medicament for myocardial protection.

Further, a unit dose of the medicament comprises iminostilbene as an active ingredient in an amount of 0.5-10 mg.

Further, the medicament is in the form of a tablet or a water injection.

In another aspect, the present invention provides a medicament for treating cardiac-cerebral ischemia-reperfusion injury, comprising iminostilbene as an active ingredient.

Further, iminostilbene is the only active ingredient.

The cardiac-cerebral vascular disease of the present invention comprises diseases caused by various reasons such as thrombus, blood rheology and vascular factors, comprising, but not limited to, thrombus, infarction, vascular rupture, etc.

The unit dose of the medicament in the present invention comprises but is not limited to a tablet, a capsule, a bag of granules or an injection, etc. according to different dosage forms.

The medicine of the present invention can be any clinically acceptable dosage forms, comprising various dosage forms for oral administration and parenteral administration. For oral administration, it can be a tablet, a capsule, a soft capsule, an oral liquid, a syrup, a granule, a dripping pill, an orally disintegrating tablet, a sustained release tablet, a sustained release capsule, a controlled release tablet and a controlled release capsule. For parenteral administration, it can be a water injection, a freeze-dried powder injection, a sterile powder injection and an infusion.

Pharmaceutically acceptable carriers or excipients in the medicament comprise but are not limited to a filler, a binder, a lubricant, a disintegrant, a cosolvent, a surfactant, an adsorption carrier, a solvent, an antioxidant, a co-solvent, an adsorbent, an osmotic pressure regulator and a PH regulator.

The medicament can further contain other traditional Chinese and western medicines for treating cardiovascular and cerebrovascular diseases, including but are not limited to superoxide dismutase, vitamin C, vitamin E, coenzyme Q10, edaravone, anti-inflammatory drugs, extract of erigeron breviscapus, danshen dripping pills, etc., or the medicament of the present application can be used in combination with those Chinese and western medicines.

The dosage forms of the present invention can be produced by any conventionally used method known to those skilled in the art of pharmaceutical preparation technology. For example, the tablets of the present invention can be prepared as follows: granulate, dry and sieve a primary medicament, an excipient, a binder, etc. by suitable methods known in the art to give a mixture, and add a lubricant, etc. thereto, then mix and prepare a tablet. Granulation can be carried out by any suitable method known in the art, such as a wet granulation method, a dry granulation method or a heating-granulation method. Suitable non-limiting examples comprise carrying out granulations with a high-speed stirring granulator, a flow granulation dryer, an extrusion granulator or a roller compactor. In addition, methods such as drying and sieving can be carried out according to the need for granulation. The mixture of the primary medicament, excipient, binder, lubricant, etc. can also be directly formed into tablets. If film coating is required, any film coating device known in the art can be used. As a film coating substrate, suitable examples includes a sugar coating substrate, a hydrophilic film coating substrate, an enteric film coating substrate and a sustained release film coating substrate.

### BRIEF DESCRIPTION OF THE DRAWINGS

FIG. 1 shows effect of ISB on myocardial infarction area in rats suffered from myocardial ischemia-reperfusion injury.

FIG. 2 shows effect of ISB on pathological changes of cardiac tissue in rats suffered from myocardial ischemia-reperfusion injury.

FIG. 3 shows effect of ISB on cardiac function in rats suffered from myocardial ischemia-reperfusion injury.

FIG. 4 shows effect of ISB on myocardial enzymes in rats suffered from myocardial ischemia-reperfusion injury.

FIG. 5 shows effect of ISB on inflammatory factors IL-1β and IL-6 in rats suffered from myocardial ischemia-reperfusion injury.

FIG. 6 shows effect of ISB on myocardial apoptosis in rats suffered from myocardial ischemia-reperfusion injury.

FIG. 7 shows effect of ISB on cerebral infarction area in rats suffered from cerebral ischemia-reperfusion injury.

### DETAILED EMBODIMENTS

The present invention will be further described in detail below with reference to specific embodiments, which does not constitute a further limitation on the present invention.

### Embodiment 1 Detection of Myocardial Infarction Area in Rats Suffered from Myocardial Ischemia-Reperfusion Injury

Animals were randomly divided into 5 groups with 10 animals in each group: a normal group, a model group, a low dose group of ISB (0.625 mg/kg), a high dose group of ISB (1.25 mg/kg), and a group treated with diltiazem hydrochloride tablet (16 mg/kg). The rats in different groups were respectively administered with different doses of ISB or diltiazem hydrochloride tablet via gavage for three continuous days prior to surgery. The rats were then ischemic for 30 min followed by reperfusion for 24 h. Then the rats were anesthetized, and their hearts were taken out by thoracotomy. Their hearts were rinsed in normal saline, placed at a temperature of -80° C. for 7 min, and taken out. Then the hearts were crosscut into 5-7 slices with a thickness of 1-2 mm along the ligature line. The slices were put into 2% TTC solution, heated in a water bath of 37° C. for 12 min, thereafter fixed in neutral formalin, and allowed to stand overnight at room temperature. Next day pictures were taken with a stereomicroscope.

The results showed that the infarcted area in the heart slices was gray in color, and the non-infarcted area in the heart slices was red in color. The infarcted area was detected by Image-Pro Plus software, and the myocardial infarction rate=infarcted area/total area of myocardial slice x100%.

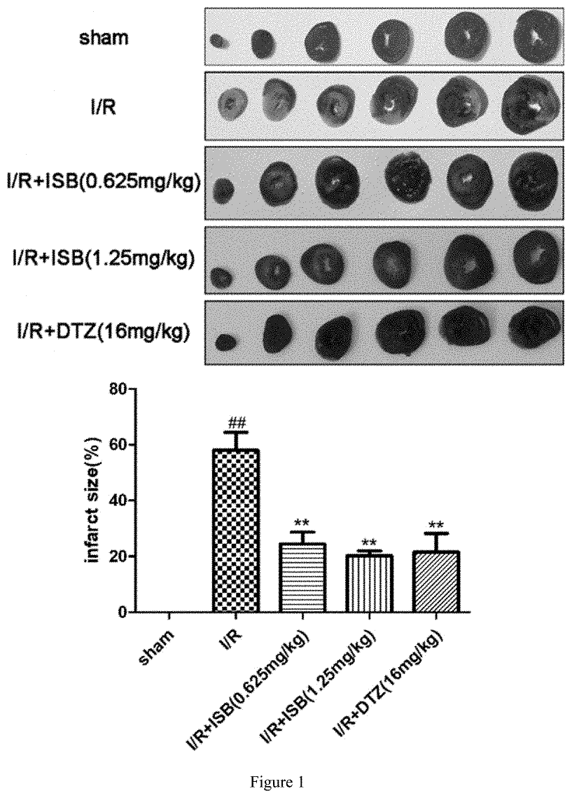

The results were shown in FIG. 1. There was no infarcted area in Sham group. The myocardial infarction area in I/R group was significantly increased as compared to Sham group. The I/R+ISB (0.625, 1.25 mg/kg) group has significantly improved myocardial tissue infarct area as compared to the I/R group.

### Embodiment 2 Effect of ISB on Pathological Changes of Cardiac Tissue in Rats Suffered From Myocardial Ischemia-Reperfusion Injury

After the experiment was completed, hearts were taken out by thoracotomy and fixed with paraformaldehyde, then paraffin sectioned and hematoxylin-eosin stained.

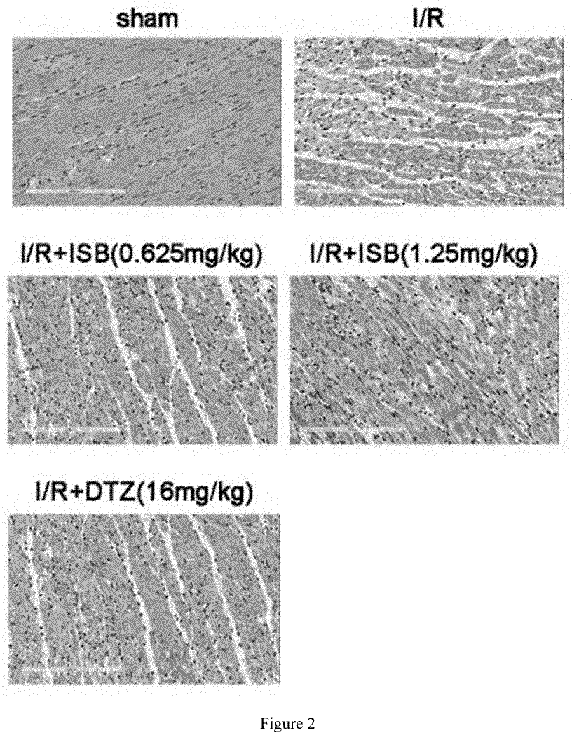

The results were shown in FIG. 2. Myocardial cells in Sham group had normal morphology and structure, intact myocardial cell membrane, normal interstitial space, and regularly arranged myocardial fibers, without inflammatory cell infiltration. However, myocardial cells in the model group had an increased interstitial space with existing of edema, obvious neutrophil infiltration, unclear direction of myocardial fibers, broken myocardial fibers in some areas and endocardial necrosis. Compared with the model group, intercellular space and inflammatory cell infiltration in the ISB groups were decreased, indicating that ISB can significantly improve the degree of myocardial injury.

### Embodiment 3 Effect of ISB on Cardiac Function in Rats Suffered from Myocardial Ischemia-Reperfusion Injury

7 days after reperfusion, the animals were anesthetized with isoflurane, and the cardiac structure and function of rats in each group were evaluated using a Visual Sonics Vevo 770 ultrasound high-resolution in vivo imaging system, and the main detection indexes were LVEF and LVFS.

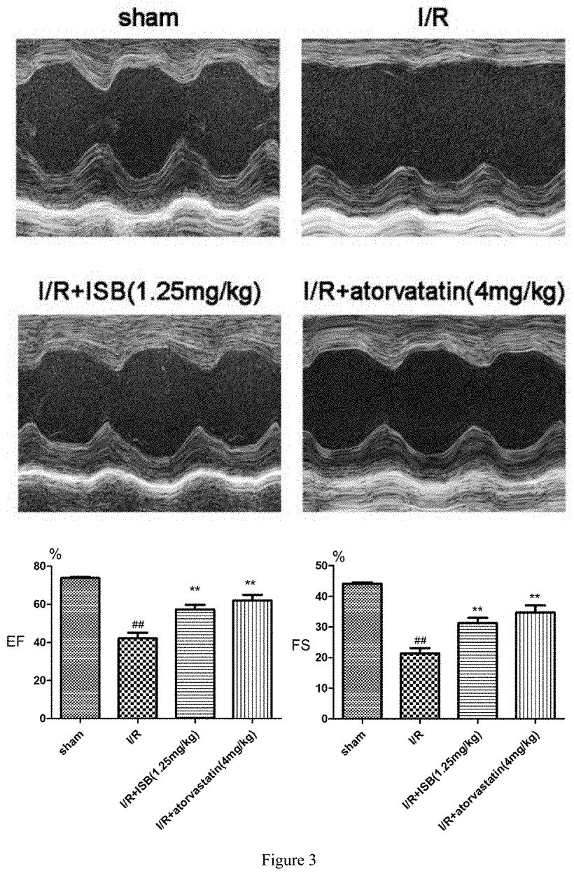

As shown in FIG. 3, compared with Sham group, EF and FS in IR model group decreased significantly, while EF and FS in ISB groups increased significantly, indicating that ISB can improve heart function.

### Embodiment 4 Effect of ISB on Myocardial Enzymes in Rats Suffered from Myocardial Ischemia-Reperfusion Injury

After reperfusion for 24 h, the rats were anesthetized with 3% pentobarbital sodium solution (30 mg/kg), and blood was taken from abdominal aorta, and centrifuged at 3500 rpm for 15 min to separate the serum. Activities of CK, LDH and AST were detected by automatic biochemical analyzer.

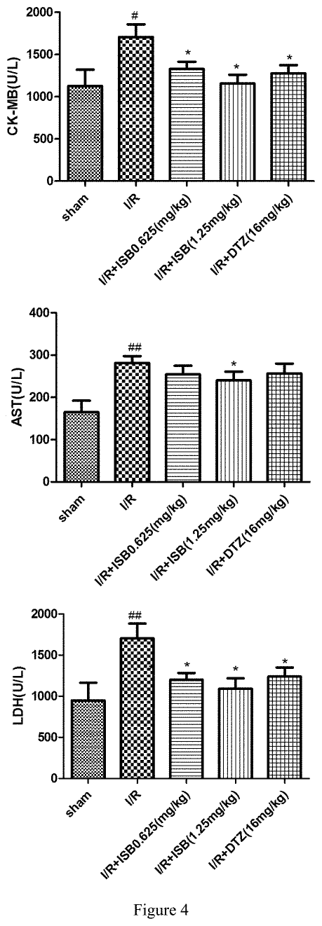

As shown in FIG. 4, compared with the Sham group, the activities of LDH, CK and AST in the serum of I/R group were significantly increased, indicating that the tissue was damaged during myocardial ischemia reperfusion. Compared with the I/R group, the leakage of LDH, CK and AST was significantly reduced in the ISB groups.

### Embodiment 5 Effect of ISB on Inflammatory Factors IL-1β and IL-6 in Rats Suffered from Myocardial Ischemia-Reperfusion Injury

After reperfusion for 24 h, the rats were anesthetized with 3% pentobarbital sodium solution (30 mg/kg), and blood was taken from abdominal aorta, and centrifuged at 3500 rpm for 15 min to separate the serum. Contents of IL-1β and IL-6 in the serum were detected according to the instructions of the kit, measured at 450 nm using a microplate reader. The contents of IL-1β and IL-6 in the serum were calculated by standard curve.

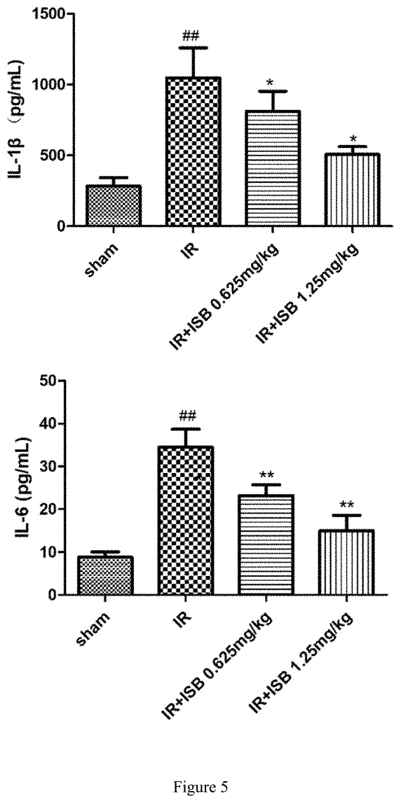

As shown in FIG. 5, compared with sham group, the contents of TNF-α and IL-6 in plasma of rats suffered from cerebral ischemia-reperfusion injury were significantly increased (P<0.01). Compared with model group, TAB (510 mg/kg) can significantly decrease the contents of TNF-α and IL-6 (P<0.05).

### Embodiment 6 Effect of ISB on Myocardial Apoptosis in Rats Suffered from Myocardial Ischemia-Reperfusion Injury

After the experiment was completed, hearts were taken by thoracotomy, fixed in paraformaldehyde, paraffin sectioned and TUNEL stained.

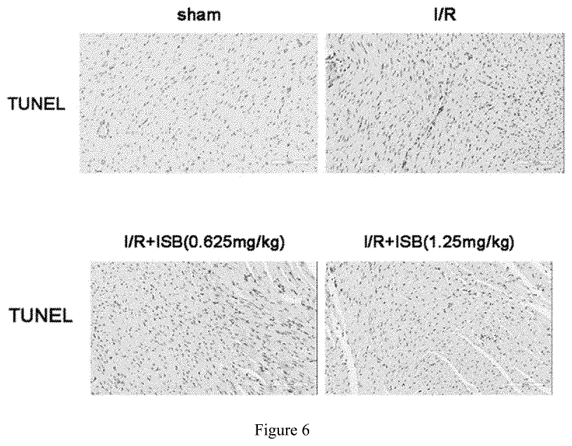

As shown in FIG. 6, TUNEL positive cells can hardly be seen in Sham group, while a large number of TUNEL positive cells can be observed in the heart of rats in IR group, and the Tunel positive myocardial cells are significantly reduced in ISB groups.

### Embodiment 7 Effect of ISB on Cerebral Infarction Area in Rats Suffered from Cerebral Ischemia-Reperfusion Injury

Animals were randomly divided into 5 groups with 10 animals in each group: a normal group, a model group, a low dose group of ISB (5 mg/kg), a high dose group of ISB (10 mg/kg), and an aspirin group (10 mg/kg). The rats in different groups were respectively administered with different doses of ISB or aspirin via gavage for three continuous days prior to surgery. The rats were then ischemia for 30 min followed by reperfusion for 24 h. Then the rats were anesthetized, and their brains were taken out. Their brains were rinsed in normal saline, placed at a temperature of −80° C. for 7 min, and taken out. Then the brains were crosscut into 5-7 slices with a thickness of 1-2 mm. The slices were put into 1% TTC solution, heated in a water bath of 37° C. for 12 min, thereafter fixed in neutral formalin, and allowed to stand overnight at room temperature. Next day pictures were taken with a stereomicroscope. The results showed that the infarcted area in the brain slices was gray in color, and the non-infarcted area in the brain slices was red in color. The infarcted area was detected by Image-Pro Plus software, and the infarction rate=infarcted area/total area of myocardial slice×100%.

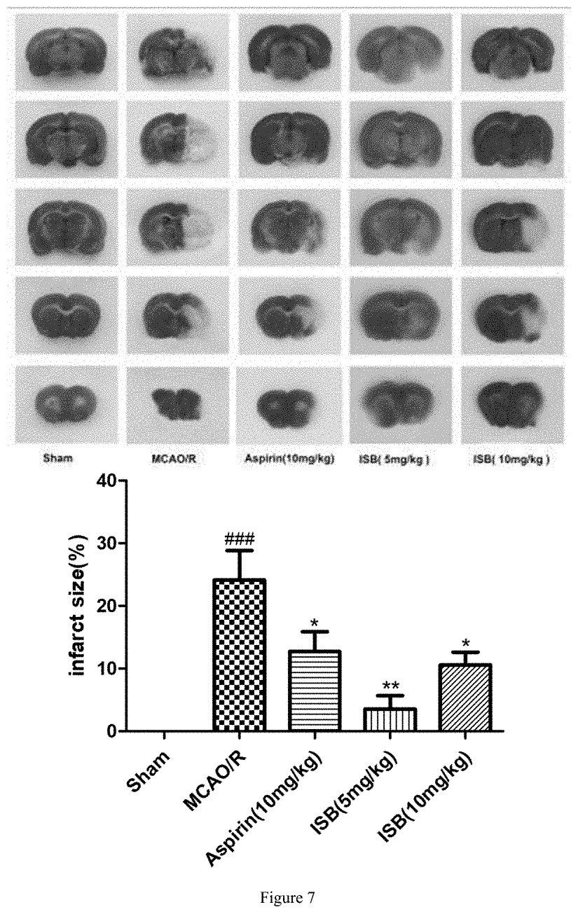

As shown in FIG. 7, compared with the Sham group, the cerebral infarction area in rats in MCAO model was significantly increased. Cerebral infarction area was significantly reduced after administration of ISB for 3 days.

It is obvious that the above-described embodiments are merely illustrative of the examples and are not intended to limit the embodiments. Other different forms of variation or modification can be made on the basis of the above description for those of ordinary skill in the art. All embodiments do not need to be exhaustive. Obvious variations or modifications are within the scope of the present invention.

1. A method medicament for treating a cardiac-cerebral vascular disease, comprising the step of administering a medicament comprising iminostilbene.

2. The method of claim 1, wherein the cardiac-cerebral vascular disease is ischemia-reperfusion injury.

3. The method of claim 2, wherein the cardiac-cerebral vascular disease is cerebral ischemia-reperfusion injury.

4. The method of claim 3, wherein the iminostilbene reduces cerebral infarction area.

5. The method of claim 2, wherein the cardiac-cerebral vascular disease is myocardial ischemia-reperfusion injury.

6. The method of claim 1, wherein the cardiac-cerebral vascular disease is ischemic heart disease.

7. The method of claim 5, wherein the iminostilbene reduces myocardial infarction area, and/or reduces LDH, AST and CK levels, and/or reduces inflammatory factors, and/or reduces myocardial apoptosis.

8. A method for myocardial protection, comprising the step of administering a medicament comprising iminostilbene.

9. The method of claim 1, wherein a unit dose of the medicament comprises iminostilbene as an active ingredient in an amount of 0.5-10 mg.

10. The method of 8 claim 1, wherein the medicament is in the form of a tablet or a water injection.

11. A medicament for treating cardiac-cerebral ischemia-reperfusion injury, comprising iminostilbene as an active ingredient.

12. The medicament of claim 11, wherein iminostilbene is the only active ingredient.

13. The method of claim 1, wherein the administering is performed orally or parenterally.

14. The method of claim 1, wherein the iminostilbene is administered at a dose of 0.625 mg/kg to 10 mg/kg, preferably 0.625 mg/kg, 1.25 mg/kg, 5 mg/kg or 10 mg/kg, for three continuous days.

15. The method of claim 8, wherein the administering is performed orally or parenterally.

16. The method of claim 1, wherein the medicament comprises pharmaceutically acceptable carriers or excipients selecting from the group consisting of a filler, a binder, a lubricant, a disintegrant, a cosolvent, a surfactant, an adsorption carrier, a solvent, an antioxidant, a co-solvent, an adsorbent, an osmotic pressure regulator, a PH regulator, and any combination thereof.

17. The method of claim 1, wherein the unit dose of the medicament is selected from the group consisting of a tablet, a capsule, a bag of granules, an injection, and any combination thereof.

18. The method of claim 6, wherein the iminostilbene reduces myocardial infarction area, and/or reduces LDH, AST and CK levels, and/or reduces inflammatory factors, and/or reduces myocardial apoptosis.

19. The method of claim 8, wherein a unit dose of the medicament comprises iminostilbene as an active ingredient in an amount of 0.5-10 mg.
